(19) United States
(12) Patent Application Publication
(10) Pub. No.: US 2021/0177343 A1
(43) Pub. Date: Jun. 17, 2021

(54) SYSTEMS AND METHODS FOR CONTACTLESS SLEEP MONITORING

(71) Applicant: Dawnlight Technologies Inc., Palo Alto, CA (US)

(72) Inventors: Erheng ZHONG, Palo Alto, CA (US); Ke ZHAI, Palo Alto, CA (US); Nan LIU, Palo Alto, CA (US); Yujia LI, Palo Alto, CA (US)

(21) Appl. No.: 17/171,361

(22) Filed: Feb. 9, 2021

Related U.S. Application Data
(63) Continuation of application No. PCT/US2020/054136, filed on Oct. 2, 2020.
(60) Provisional application No. 62/910,323, filed on Oct. 3, 2019.

(51) Int. Cl. A61B 5/00; A61B 5/01; A61B 5/024; A61B 5/11; A61B 5/08

(52) U.S. Cl. CPC A61B 5/4812; A61B 5/7267; A61B 5/725; A61B 5/01; A61B 5/0823; A61B 5/024; A61B 5/11; A61B 5/74; A61B 5/4839; A61B 5/0008

(57) ABSTRACT

Disclosed herein are systems and methods for contactless sleep monitoring. The contactless sleep monitoring system collects patient data from a plurality of sensors, including thermal, radar, and audio sensors. The data is then processed using various signal processing techniques. Machine learning algorithms then convert the thermal data, audio data, and radar data into latent representations, preserving the features of each type of data but enabling them to be combined together for analysis. Finally, the system fuses the representations and then predicts sleep states by performing machine learning analysis on the fused data. Sleep states include sleep stages and sleep conditions.

### CROSS-REFERENCE

This application is a continuation of International Application No. PCT/US2020/054136, filed Oct. 2, 2020, which claims the benefit of U.S. Provisional Application No. 62/910,323, filed Oct. 3, 2019, each of which is incorporated by reference herein in its entirety.

### BACKGROUND

Currently, many systems that monitor sleep conditions (e.g., sleep apnea, restless leg syndrome, insomnia, or interrupted sleep) include sensors attached to a person's body. The invasive sensors used by existing systems are often expensive, inconvenient for use by health care providers, and uncomfortable for patients. Although commercial sleep tracking is available on consumer devices (e.g., smartwatches), these devices may not be able to detect sleep conditions using methods that are reliable and usable by health care providers to decide to proceed with escalations of care.

### SUMMARY

There exists a need for a noncontact method of monitoring and diagnosing sleep conditions without significantly introducing patient discomfort or requiring a hospital visit. Unlike existing systems that monitor patients and provide similar health screening capabilities, the disclosed system may be deployed either in a care facility (e.g., a hospital), or in a patient's home. The system uses sensors that collect data remotely, not requiring the patient to be physically connected to any devices or sensors. Instead, the system may passively collect data while the patient sleeps uninterrupted. Additionally, by using contactless sensors not present in consumer devices (e.g., smartwatches), the disclosed system may be able data that is usable in a clinical setting (e.g., is reliable for health care providers).

In an aspect, a method for electronically outputting a sleep state of a subject, is disclosed. The method comprises (a) obtaining a plurality of signals sensed from the subject using a plurality of sensors, wherein the plurality of signals comprises at least two signals selected from the group consisting of a radar signal, a thermal signal, and an audio signal, (b) computer processing the plurality of signals to generate a latent representation of at least a subset of the plurality of signals obtained in (a), (c) generating a fused data set based at least in part on the latent representation generated in (b), (d) using a trained algorithm to process the fused data set generated in (c) to generate a sleep state of the subject. and (e) electronically outputting the sleep state of the subject determined in (d).

In some embodiments, the plurality of signals comprises a radar signal, a thermal signal, and an audio signal.

In some embodiments, the trained algorithm comprises a trained machine learning classifier.

In some embodiments, the trained algorithm is selected from the group consisting of a recurrent neural network, a convolutional neural network, a decision tree, a logistic regression, a support vector machine, and any combination thereof.

In some embodiments, the plurality of sensors comprises a radar antenna that senses the radar signal.

In some embodiments, the radar signal is a range-doppler signal.

In some embodiments, the range-doppler signal is sensed using an intelligent millimeter-wave (mmWave) sensor or an IR-UWB radar.

In some embodiments, the (b) comprises performing at least one signal processing operation on the radar signal, wherein the signal processing operation is selected from the group consisting of phase unwrapping, beamforming, clutter removal, adaptive filtering, bandpass filtering, spectrum estimation, calculating a phase differential, phase mapping, and any combination thereof.

In some embodiments, (b) comprises performing the spectrum estimation, wherein the spectrum estimation produces an estimated heart rate or an estimated respiration rate of the subject.

In some embodiments, (b) comprises performing the phase differential, wherein the phase differential produces a motion measurement of the subject.

In some embodiments, (b) comprises performing the phase mapping, wherein the phase mapping produces a respiratory tidal measurement of the subject.

In some embodiments, the plurality of sensors comprises an infrared camera that senses the thermal signal and provides one or more thermal images for the computer processing.

In some embodiments, (b) comprises performing at least one signal processing operation on the thermal signal selected from the group consisting of equalization, reshaping, normalization, and any combination thereof.

In some embodiments, the 13 further comprises, subsequent to performing the at least one signal processing operation on the thermal signal in (b), using representation learning to perform face detection based at least in part on the latent thermal representation of the thermal signal.

In some embodiments, the face detection generates at least one of a position measurement, a temperature measurement, an airflow measurement, and any combination thereof.

In some embodiments, the face detection comprises generating the position measurement, wherein generating the position measurement comprises at least one of landmark detection, pose estimation, and any combination thereof.

In some embodiments, the face detection comprises generating the temperature measurement, wherein generating the temperature measurement comprises at least one of forehead detection, temperature extraction, and any combination thereof.

In some embodiments, the face detection comprises generating the airflow measurement, wherein generating the airflow measurement comprises at least one of nose detection, temperature change detection, and any combination thereof.

In some embodiments, the plurality of sensors comprises a microphone that senses the audio signal.

In some embodiments, (b) comprises performing at least one signal processing operation on the audio signal selected from the group consisting of resampling, applying a bandpass filter, applying a mel-spectrum transform, and any combination thereof.

The method of claim 20, subsequent to performing the at least one signal processing operation on the audio signal in (b), using representation learning to generate at least one of a cough amplitude, a cough frequency, a snoring amplitude, a snoring duration, and any combination thereof, based at least in part on the latent audio representation of the audio signal.

In some embodiments, the representation learning generates the cough amplitude or the cough frequency, wherein generating the cough amplitude or the cough frequency comprises performing cough detection on the latent audio representation of the audio signal.

In some embodiments, the representation learning generates the snoring amplitude or the snoring duration, wherein generating the snoring amplitude or the snoring duration comprises performing snoring detection on the latent audio representation of the audio signal.

In some embodiments, (c) further comprises fusing physiological data of the subject.

In some embodiments, psychology data comprises vital sign data, motion data, position data, audio event data, or a combination thereof of the subject.

In some embodiments, the vital sign data comprises at least one vital sign selected from the group consisting of respiration rate, tidal volume, nasal airflow, pulse rate, body temperature, motion data, position data, seated position, standing position, supine position, prone position, and audio event data.

In some embodiments, the sleep state comprises a sleep stage.

In some embodiments, the sleep stage is selected from the group consisting of wake, rapid eye movement (REM) sleep, and non-REM sleep.

In some embodiments, the sleep state comprises a sleep condition or a sleep disorder.

In some embodiments, the sleep condition or the sleep disorder is selected from the group consisting of sleep apnea, insomnia, restless leg syndrome, interrupted sleep, and any combination thereof.

In some embodiments, the 1 further comprises generating a notification based at least in part on the sleep state of the subject.

In some embodiments, the notification is presented to a user.

In some embodiments, the user is the subject or a health care provider of the subject.

In some embodiments, the 29 further comprises administering a treatment to the subject for the sleep condition or the sleep disorder.

In some embodiments, the treatment comprises one or more members selected from the group consisting of administering melatonin, administering a sedative, and administering a sleep therapy.

In an aspect, a system for electronically outputting a sleep state of a subject is disclosed. The system comprises a plurality of sensors comprising at least two members selected from the group consisting of a radar sensor, a thermal sensor, and an audio sensor. The system also comprises a computation unit comprising circuitry configured to: (i) computer process a plurality of signals sensed from a subject using the at least two members selected from the group consisting of the radar sensor, the thermal sensor, and the audio sensor, to generate a latent representation of at least a subset of the plurality of signals; (ii) generating a fused data set based at least in part on the latent representation generated in (i); (iii) using a trained algorithm to process the fused data set generated in (ii) to generate a sleep state of the subject; and (iv) electronically output the sleep state of the subject determined in (iii).

In another aspect, a method for electronically outputting a sleep state of a subject is disclosed. The method comprises (a) obtaining a plurality of signals sensed from the subject using a plurality of sensors. The plurality of signals comprises a radar signal, a thermal signal, and an audio signal. The method further comprises b) computer processing the plurality of signals to generate a latent representation of at least a subset of the plurality of signals obtained in (a). The method further comprises (c) generating a fused data set based at least in part on the latent representation generated in (b). The method further comprises (d) using a trained machine learning classifier to process the fused data set generated in (c) to generate a sleep state of the subject. Finally, the method further comprises (e) electronically outputting the sleep state of the subject determined in (d).

In some embodiments, (a) comprises using the plurality of sensors to sense the plurality of signals.

In some embodiments, the plurality of sensors comprises a radar sensor, a thermal sensor, and an audio sensor, wherein the radar sensor, the thermal sensor, and the audio sensor are included with a same device.

In some embodiments, the machine learning classifier is a multilayer perceptron (MLP) or a recurrent neural network (RNN).

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates a diagram of the contactless sleep monitoring system, in accordance with an embodiment;

FIG. 2 illustrates a diagram of the computation unit of FIG. 1;

FIG. 3 illustrates a radar processing layer, in accordance with an embodiment;

FIG. 4 illustrates a thermal processing layer, in accordance with an embodiment;

FIG. 5 illustrates an audio processing layer, in accordance with an embodiment;

FIG. 6 illustrates a sensor fusion layer, in accordance with an embodiment; and

FIG. 7 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The disclosed system performs sleep monitoring by processing fused data (e.g., vital sign data) with machine learning algorithms. The system may collect sleep data using a plurality of contactless sensors, apply signal processing techniques to the collected data in order to enhance the signals collected from the sensors, perform machine learning to develop representations of the sensor data, fuse the representations of the sensor data, and produce predictions of sleep states. Sleep states may include sleep conditions, such as sleep apnea, or sleep stages, including awake, rapid eye movement (REM) sleep, and non-REM sleep.

The disclosed system includes a plurality of sensors to measure vital signs, including audio sensors, thermal sensors, and radar sensors. The audio sensors may be microphones. The thermal sensors may be infrared cameras. The sensors used by the sleep system may be contactless, to ensure the patient does not feel his or her privacy or personal space is invaded. Using non-contact sensing may make the system non-intrusive and easy to set up in, for example, a home environment for long term continuous monitoring. Using a machine learning based sensor fusion approach may produce accurate measurements without requiring expensive devices such as EEGs. Also, from the perspective of compliance with health standards, the contactless sleep monitoring system may require minimal to no effort by a patient to install and operate the system, making it easier to comply with FDA regulations.

Signal processing techniques may be used to enhance the signal data once it is captured by the sensors. Generally, the signal processing techniques may be techniques to improve the signal strength, by removing cluttering and amplifying aspects of the signals salient to monitoring sleep. Additional signal processing techniques may produce representations of the data, including signal power representations, to determine frequencies associated with bodily functions or sounds indicative of sleep conditions or sleep states.

Representation learning creates representations of the sensor data that the system can use to fuse the different forms of sensor data together. Representation learning may include reconfiguring the sensor data into a format in which it may be combined with data from other sensors, creating sensor latent representations. These representations may preserve the feature content of the data provided by the sensors, in order for the system to perform machine learning analysis on the combined data. After fusing the data, the machine learning analysis may produce predictions of sleep states, including sleep stages and sleep conditions.

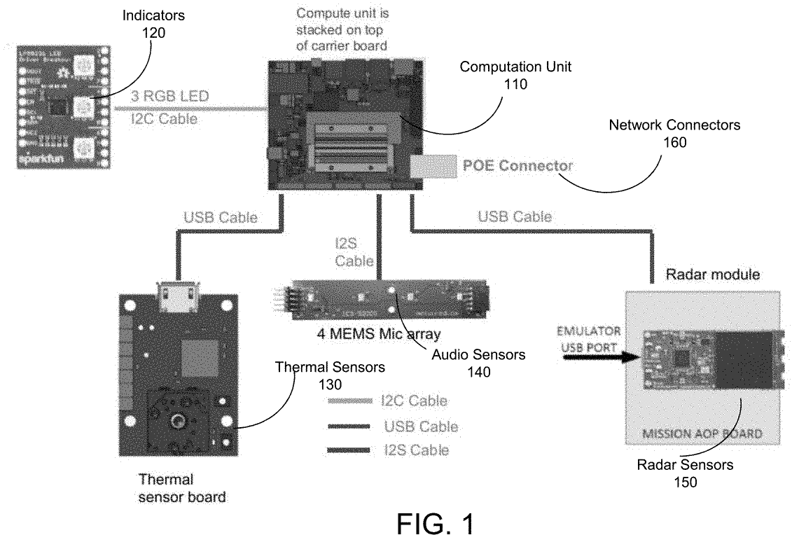

FIG. 1 schematically illustrates a diagram of a contactless sleep monitoring system 100, in accordance with an embodiment of the disclosure. The contactless sleep monitoring system 100 is configured to monitor and diagnose one or more sleep states associated with a user. The contactless sleep monitoring system 100 includes a computation unit 110, one or more thermal sensors 130, one or more radar sensors 150, one or more audio sensors 140, and one or more indicators 120.

Generally, the sensors may be configured to remotely measure and generate data associated with bodily functions of the user, in a contact-free manner. For example, the sensors may generate sets of quantitative data associated with measurements of body functions including breathing processes and respiration processes, coughs, snores, expectorations, and wheezes.

The computation unit 110 may process the sets of quantitative data to generate diagnoses of sleep conditions or predictions of sleep states. The computation unit 110 may include a signal processing module to modify the received signal data to provide enhanced signal data for analysis. A machine learning module may then perform machine learning analysis on the signal-processed data, to generate predictions of sleep states. The data processed may include current or substantially real-time sensor data, historical data, or a combination thereof.

The thermal sensors 130 may collect information about the user's body temperature at various locations on the user's body during sleep. The thermal sensors 130 may be infrared cameras configured to capture infrared images of the user's body during sleep. The images from the thermal sensors 130 may be analyzed using a machine learning algorithm, such as a convolutional neural network (CNN), to determine thermal features indicative of sleep stages or sleep conditions.

The radar sensors 150 may remotely perform ranging and detection functions associated with bodily functions such as respiration. The radar sensors 150 may be arranged in an array. The radar sensors 150 may be radar antennae. The radar may be a millimeter wave (mmWave) or an IR-UWB radar designed for indoor use. The radar sensors 150 may be capable of capturing fine motions of a user including the user's breathing. The radar may be configured to sense a range-doppler signal.

The audio sensors 140 may be configured to remotely sense sounds including coughs, snores, wheezes, or expectorations. The audio sensors 140 may be microphones configured to capture audio data from a user. The audio sensors 140 may include multiple regions from which to collect input audio data from a user (e.g., mouth, nose, trunk, legs).

The indicators 120 may be configured to provide alerts to the user or medical personnel regarding sleep conditions or sleep stages. The indicators 120 may be light-emitting diodes configured to flash to warn the user or medical professionals of distressing sleep events. The indicators may also provide sound alarms to inform the user or medical professionals of conditions needing urgent care. Sleep apnea detection results may be reported to the user for reference.

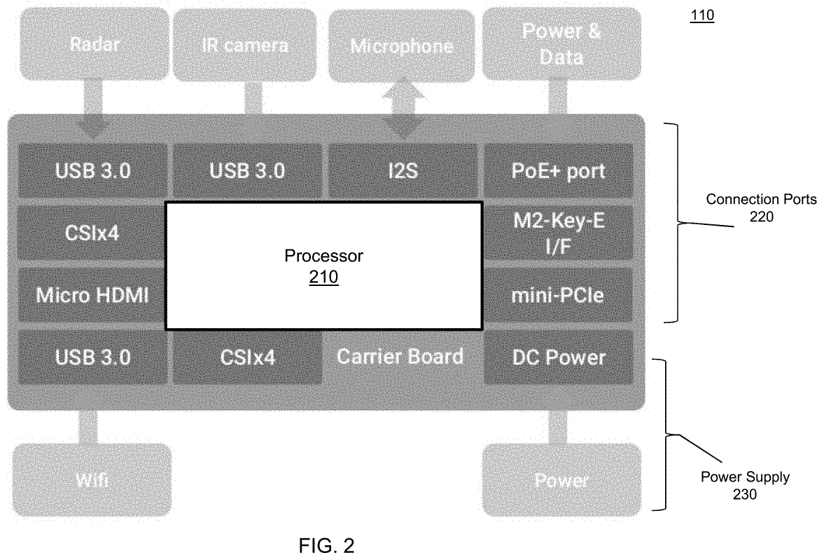

FIG. 2 illustrates a diagram of the computation unit 110. The computation unit 110 includes a power supply 230, connection ports 210, and a processor 210.

The connection ports 210 are configured to manage communication protocols and associated communication with external peripheral devices (e.g., the thermal sensors 130, radar sensors 150, audio sensors 140, and input devices such as keyboards and mice) as well as communication with other components in the computation unit 110. The connection ports 210 may be universal serial bus (USB) ports, HDMI ports, and network connection ports 210. The connection ports 210 may be configured to interface the computation unit 110 with one or more external devices such as an external hard drive, an end user computing device (e.g., a laptop computer or a desktop computer), and so on. The connection ports 210 may include sensor interfaces configured to implement necessary communication protocols that allow the processor 210 to receive the sensor data.

The processor 210 may perform the signal processing and machine learning computations for sleep state prediction. The processor 210 may be an artificial intelligence (AI) accelerator. The processor 210 may be a graphic processing unit (GPU), fixed-programmable gate array (FPGA), or tensor processing unit (TPU). The processor 210 may process the quantitative data using one or more machine learning algorithms such as neural networks, linear regression, a support vector machine, or the like.

The computation unit 110 may include a memory, including both short-term memory and long-term memory. The memory may be used to store, for example, substantially real-time and historical quantitative data sets generated by the sensors. The memory may be comprised of any combination of hard disk drives, flash memory, random access memory, read-only memory, solid state drives, and other memory components.

The power supply 230 may supply a direct current (DC) voltage or supply power over Ethernet (POE) to the computation unit 110 in order to enable performance of calculations. The power supply 230 may also be used to power one or more of the sensors 130, 140, and 150. The sensors may alternatively use their own power supplies.

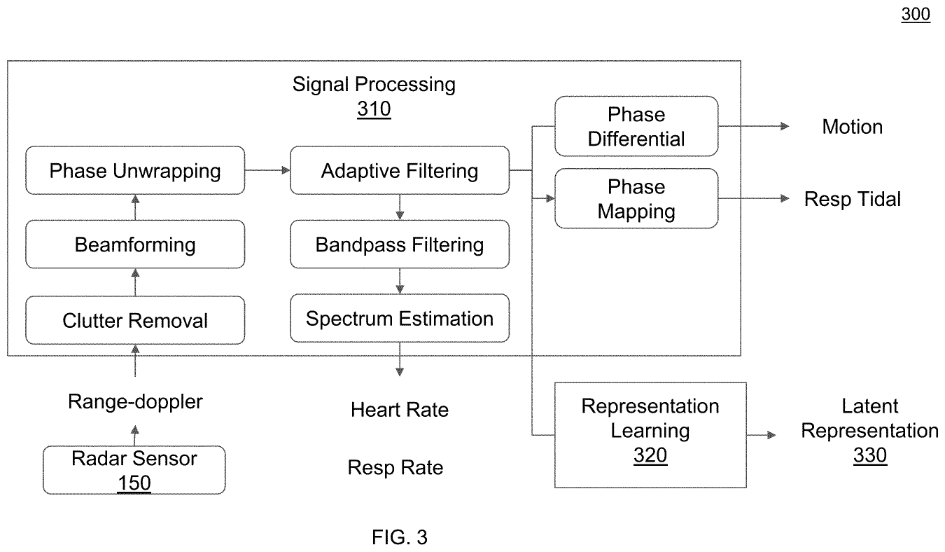

FIG. 3 illustrates a radar processing layer 300, in accordance with an embodiment. The radar processing layer 300 receives input data from a radar sensor, performs signal processing 310 to produce additional inputs for data fusion, and creates a radar representation for fusion with a thermal representation, an audio representation, or both.

In FIG. 3, the radar processing layer 300 may perform signal processing 310 in the following sequence: clutter removal, beamforming, phase unwrapping, and adaptive filtering. In this disclosure, a layer refers to a set of related processes executing on the processor. For example, a signal processing layer may include various filtering methods, while a machine learning layer may include several machine learning algorithms executed in sequence. Following adaptive filtering, the system 100 may estimate a heart rate and a respiration rate from the processed radar data by after performing bandpass filtering and spectrum estimation following adaptive filtering. Additionally, following adaptive filtering, the system 100 may calculate a phase differential to analyze body motion and phase mapping to measure tidal breathing. The adaptively filtered signal may be further processed by a representation learning 320 for radar data block to create a radar latent representation 330 of the radar data. The radar processing layer 300 may perform phase unwrapping to overcome phase discontinuities, enabling the system to perform additional signal processing operations (e.g., bandpass filtering).

In embodiments, processing data generated by radar includes one or more signal processing operations. Processing data generated by radar may involve background modeling and removal. In the embodiment of FIG. 3, background clutter may be mostly static and can be detected and removed using, for example, a moving average. The moving average may be produced by averaging signal strengths over successive time periods. Clutter removal may remove a direct current (DC) offset from the signal. Multiple radar antennas in a radar sensor 150 may be arranged in such a configuration to enable beamforming, when radar signals transmitted from individual radar antennae constructively interfere to enhance the generated radar signal from the radar sensor configuration. The system 100 may remove random body motions using adaptive filters, such as a Kalman filter. The system 100 may use bandpass filtering to separate heartbeat and respiration components from the radar sensor data. The system 100 may perform time frequency analysis on the sensor data using a wavelet transform and a short-time Fourier transform to produce a spectrogram. Spectrum estimation enables the system 100 to determine bodily functions, such as heart rate and respiration rate, by forming a representation of the power spectral density of the reflected radar signals and extracting feature information from this alternate representation of the signal. To determine body motion, the system 100 may calculate a phase differential between the transmitted radar signal and the reflected radar signal. Tidal volume of breathing may be estimated by mapping the phase differences to distance changes using an equation (λ/4πT)Δθ, where λ is the wavelength of the radar sensor, T is the time gap between two phases and Δθ is the phase difference.
Machine learning algorithms may process the spectrogram to predict the heart rate and respiratory rate from the radar sensor data. In some embodiments, the machine learning algorithms include any combination of a neural network, a linear regression, a support vector machine, and any other machine learning algorithm(s).

The structure described above can be extended to detect other kinds of motion associated with the user, such as shaking.

The representation learning 320 for radar data may use machine learning to create a latent radar representation 330, reconfiguring the processed sensor data into a form that preserves the unique features of the data and enables it to be fused with either the thermal data or the audio data, or both. Representation learning may include removing information about extraneous attributes of the data that are not features analyzed by the machine learning algorithms (compression). [[What ML

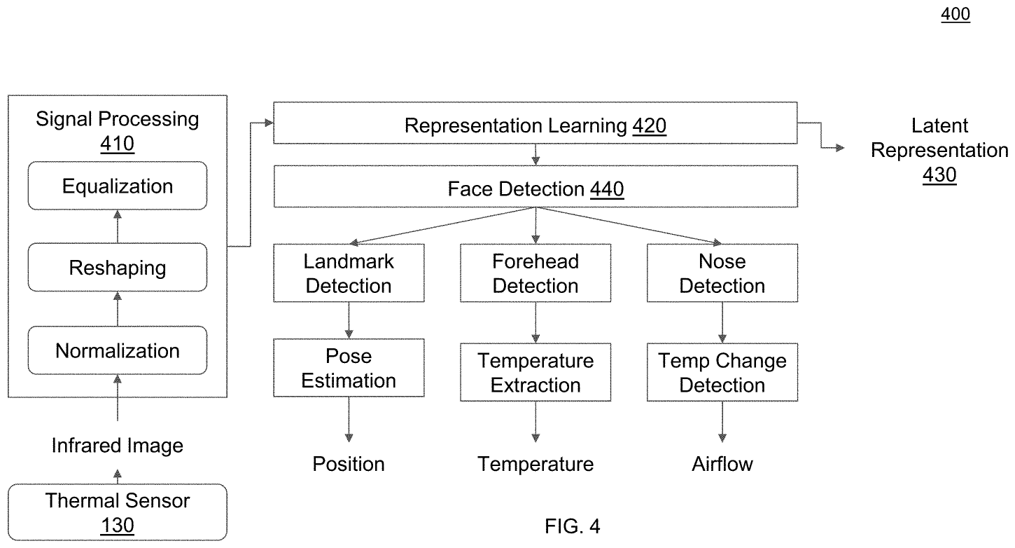

FIG. 4 illustrates a thermal processing layer 400. The thermal processing layer 400 may receive input data from a thermal sensor, may perform signal processing 410 to produce additional inputs for data fusion, and may create a thermal representation.

In the thermal processing layer 400, the system 100 may perform signal processing 410 in a sequence in accordance with the embodiment of FIG. 4. For example, the system 100 may perform normalization, reshaping, and equalization on an infrared image produced by the thermal sensors 130 (e.g., infrared cameras). The signal-processed thermal data may be further processed using a representation learning 420 for thermal data algorithm, to create a thermal latent representation 430.

Normalization may change the amplitude of the received thermal signal in order to increase the signal strength of areas of interest. Reshaping may change the thermal image into proper size for face detection models. Equalization may reduce distortion in the thermal image, making it easier for the machine learning algorithm to analyze features relevant to sleep state prediction.

After performing representation learning 420 for thermal data, the thermal latent representation 430 may be used to perform face detection 440. Face detection 440 may include position detection, body temperature detection, and airflow analysis. The system 100 may perform face detection using an eigen-face technique, an object detection framework (such as the Viola-Jones object detection framework), or a neural network, such as a convolutional neural network, to determine predictions for position based on orientations of specific features or temperature based on colors or shades in an infrared photo, for example. The system 100 may perform position detection by first performing landmark detection and then pose estimation. Landmark detection may determine where on the face specific features are located, and then pose estimation may determine the gaze direction and orientation of the user's face. The system 100 may perform temperature detection by first performing forehead detection and temperature extraction to determine the temperature of the user's forehead and relate the determined temperature to the human's body temperature. For example, a forehead temperature may be predictably lower than an oral temperature, e.g., by 0.5° F. (0.3° C.) to 1° F. (0.6° C.). The airflow detection may be performed using nose detection and then temperature change detection. Nose detection may locate the user's nose, while the temperature change detection may determine the change in temperature of regions near the nostrils, allowing the airflow to be detected.

The representation learning 420 for thermal data stage may use machine learning to create a latent space representation, reconfiguring the processed sensor data into a form that preserves the unique features of the data and enables it to be fused with either the radar data or the audio data, or both.

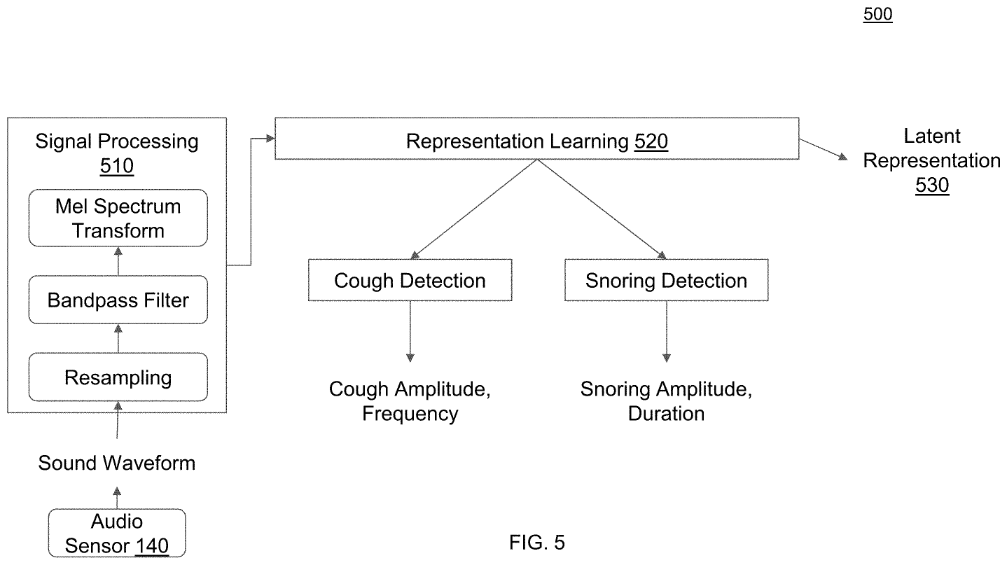

FIG. 5 illustrates an audio processing layer 500, in accordance with an embodiment. The thermal processing layer 400 receives input data from one or more audio sensors 140, performs signal processing 510 to produce additional inputs for data fusion, and creates a latent audio representation.

The audio processing layer 500 may perform signal processing 510 on the audio signal received through the microphone. The audio signal may be a sound waveform. The system 100 may perform resampling (to reduce the processing cost of computation), bandpass filtering, and a mel-spectrum transform to process the signal. The mel-spectrum transform may make auditory features more prominent, as performing mel-spectrum transforms closely approximates a human's auditory system 100 response. Bandpass filtering may better isolate sounds associated with sleep states (e.g., coughing, wheezing, and snoring). The signal-processed audio data may be analyzed by a representation learning 520 for audio data algorithm. The latent audio representation 530 may be processed to determine cough amplitude and frequency using a cough detection algorithm, and snoring amplitude and duration may be predicted using a snoring detection algorithm.

The representation learning 520 for audio data stage may use machine learning to create a latent space representation, reconfiguring the processed sensor data into a form that preserves the unique features of the data and enables it to be fused with either the radar data or the thermal data, or both.

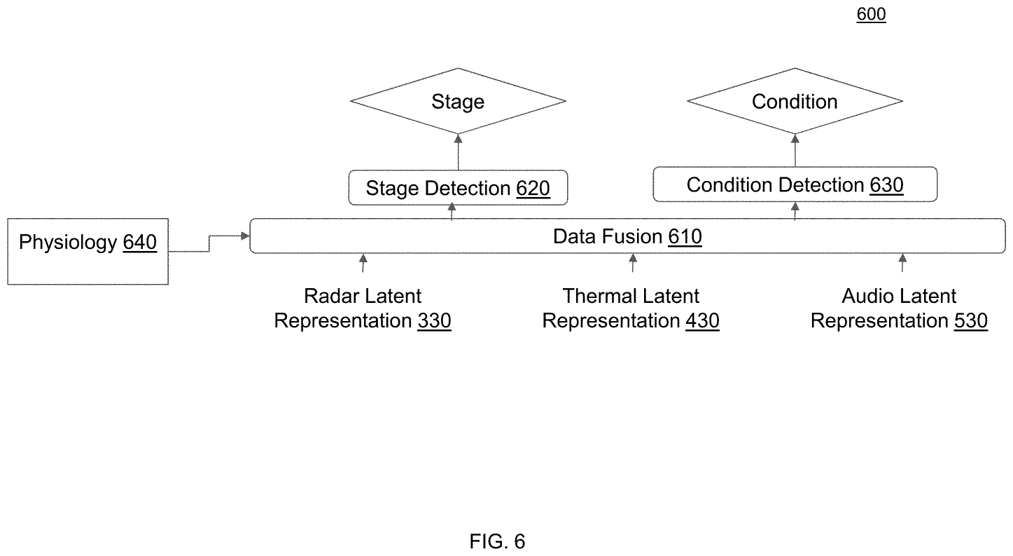

FIG. 6 illustrates a sensor fusion layer 600, in accordance with an embodiment. The sensor fusion layer 600 combines the audio, thermal, and radar representations into fused data. Then, the sensor fusion layer 600 uses machine learning to detect one or more sleep states.

The data fusion layer 610 processes a combination of representations from the thermal sensors 130, radar sensors 150, and audio sensors 140. The fusion layer may merge the representations together, for example, by concatenation, pooling, computing a product, or by another method, train classifiers on the concatenated representations, and produce predictions using the trained classifiers. The fusion layer may include multiple classifiers (e.g., a sleep apnea classifier, a multiclass sleep state classifier) configured to receive at least two of the thermal latent representation 430, the audio latent representation 530, and the radar latent representation 330. In some embodiments, outputs produced by the sensors are processed in real time in order to provide real time alerts. In other embodiments, historical data and statistics are used to predict the sleep states. In still other embodiments, the contactless sleep monitoring system 100 is configured to use a combination of real-time data and historical data generated by the sensors to predict the sleep states. Additionally, the data fusion layer 610 may incorporate and analyze physiology data 640, which may include vital sign measurements collected by the sensors as well as intermediate predictions made (e.g., motion, position, and audio event data). The physiology data 640 may also be placed in a representation before being incorporated in the data fusion layer 610.

Using a sensor fusion approach may enable a greater confidence level in detecting sleep states associated with a user. Using a single sensor may increase a probability associated with incorrect predictions, especially when there is an occlusion, a blind spot, a long range or multiple people in a scene as observed by the sensor. Using multiple sensors in combination and combining data processing results from processing discrete sets of quantitative data generated by the various sensors may produce a more accurate prediction, as different sensing modalities may complement each other in their capabilities.

The stage detection layer 620 and condition detection layer 630 use machine learning algorithms to produce predictions of sleep states. The classifiers may be binary or multiclass classifiers. For example, the system 100 may use binary classifiers to determine the presence of a sleep disorder, such as sleep apnea, insomnia, disturbed sleep, or restless leg syndrome. With respect to sleep stages, the system 100 may use a multiclass classifier, to predict whether the user is in REM, non-REM sleep, deep sleep, or awake. The algorithms may trained by analyzing ground truth data from sleep measurement devices (e.g., polysomnography (PSG) devices) collecting data from a control group (people without sleep disorders) and an experimental group (e.g., people with sleep apnea). The classifiers used may use algorithms including decision trees, support vector machines, neural networks (including convolutional and recurrent neural networks (CNNs and RNNs), such as long short-term memory (LSTM networks), logistic regressions, or a combination thereof.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Computer Systems

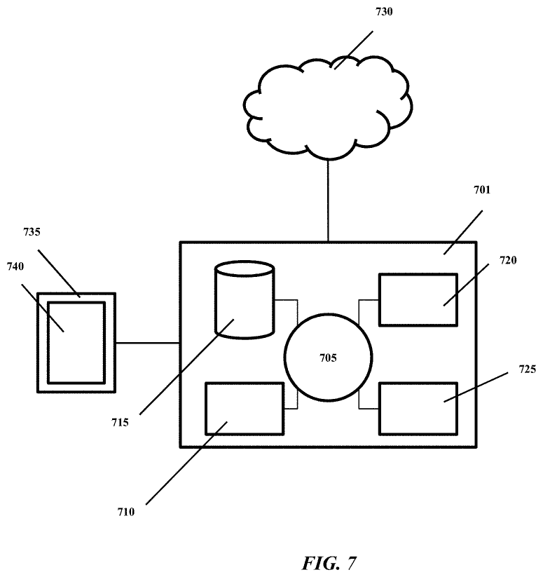

The present disclosure provides computer systems that are programmed to implement methods of the disclosure. FIG. 7 shows a computer system 701 that is programmed or otherwise configured to perform signal processing, fuse sensor data, and perform machine learning operations. The computer system 701 can regulate various aspects of contactless sleep monitoring of the present disclosure, such as, for example, performing machine learning tasks The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some cases is a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, a method for configuring machine learning algorithms. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, create a latent representation of sensor data.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method for electronically outputting a sleep state of a subject, comprising:
(a) obtaining a plurality of signals sensed from said subject using a plurality of sensors, wherein said plurality of signals comprises at least two signals selected from the group consisting of a radar signal, a thermal signal, and an audio signal;
(b) computer processing said plurality of signals to generate a latent representation of at least a subset of said plurality of signals obtained in (a);
(c) generating a fused data set based at least in part on said latent representation generated in (b);
(d) using a trained algorithm to process said fused data set generated in (c) to generate a sleep state of said subject; and
(e) electronically outputting said sleep state of said subject determined in (d).

2. The method of claim 1, wherein said plurality of signals comprises said radar signal, said thermal signal, and said audio signal.

3. The method of claim 1, wherein said trained algorithm comprises a trained machine learning classifier.

4. The method of claim 1, wherein said trained algorithm is selected from the group consisting of a recurrent neural network, a convolutional neural network, a decision tree, a logistic regression, a support vector machine, and any combination thereof.

5. The method of claim 1, wherein said plurality of sensors comprises at least one of a radar antenna that senses said radar signal, a microphone that senses said audio signal, and an infrared camera that senses said thermal signal and provides one or more thermal images for said computer processing.

6. The method of claim 1, wherein said radar signal is a range-doppler signal.

7. The method of claim 1, wherein said (b) comprises performing at least one signal processing operation on said radar signal, wherein said signal processing operation is selected from the group consisting of phase unwrapping, beamforming, clutter removal, adaptive filtering, bandpass filtering, spectrum estimation, calculating a phase differential, phase mapping, and any combination thereof.

8. The method of claim 7, wherein (b) comprises performing said spectrum estimation, wherein said spectrum estimation produces an estimated heart rate or an estimated respiration rate of said subject.

9. The method of claim 7, wherein (b) comprises performing said phase differential, wherein said phase differential produces a motion measurement of said subject.

10. The method of claim 7, wherein (b) comprises performing said phase mapping, wherein said phase mapping produces a respiratory tidal measurement of said subject.

11. The method of claim 1, wherein (b) comprises performing at least one signal processing operation on said thermal signal selected from the group consisting of equalization, reshaping, normalization, and any combination thereof.

12. The method of claim 11, further comprising, subsequent to performing said at least one signal processing operation on said thermal signal in (b), using representation learning to perform face detection based at least in part on said latent thermal representation of said thermal signal.

13. The method of claim 12, wherein said face detection generates at least one of a position measurement, a temperature measurement, an airflow measurement, and any combination thereof.

14. The method of claim 13, wherein said face detection comprises generating said position measurement, wherein generating said position measurement comprises at least one of landmark detection, pose estimation, and any combination thereof.

15. The method of claim 13, wherein said face detection comprises generating said temperature measurement, wherein generating said temperature measurement comprises at least one of forehead detection, temperature extraction, and any combination thereof.

16. The method of claim 13, wherein said face detection comprises generating said airflow measurement, wherein generating said airflow measurement comprises at least one of nose detection, temperature change detection, and any combination thereof.

17. The method of claim 1, wherein (b) comprises performing at least one signal processing operation on said audio signal selected from the group consisting of resampling, applying a bandpass filter, applying a mel-spectrum transform, and any combination thereof.

18. The method of claim 17, subsequent to performing said at least one signal processing operation on said audio signal in (b), using representation learning to generate at least one of a cough amplitude, a cough frequency, a snoring amplitude, a snoring duration, and any combination thereof, based at least in part on said latent audio representation of said audio signal.

19. The method of claim 18, wherein said representation learning generates said cough amplitude or said cough frequency, wherein generating said cough amplitude or said cough frequency comprises performing cough detection on said latent audio representation of said audio signal.

20. The method of claim 18, wherein said representation learning generates said snoring amplitude or said snoring duration, wherein generating said snoring amplitude or said snoring duration comprises performing snoring detection on said latent audio representation of said audio signal.

21. The method of claim 1, wherein (c) further comprises fusing physiological data of said subject.

22. The method of claim 21, wherein psychology data comprises vital sign data, motion data, position data, audio event data, or a combination thereof of said subject.

23. The method of claim 1, wherein said vital sign data comprises at least one vital sign selected from the group consisting of respiration rate, tidal volume, nasal airflow, pulse rate, body temperature, motion data, position data, seated position, standing position, supine position, prone position, and audio event data.

24. The method of claim 1, wherein said sleep state comprises a sleep stage.

25. The method of claim 24, wherein said sleep stage is selected from the group consisting of wake, rapid eye movement (REM) sleep, and non-REM sleep.

26. The method of claim 1, wherein said sleep state comprises a sleep condition or a sleep disorder.

27. The method of claim 26, wherein said sleep condition or said sleep disorder is selected from the group consisting of sleep apnea, insomnia, restless leg syndrome, interrupted sleep, and any combination thereof.

28. The method of claim 1, further comprising generating a notification based at least in part on said sleep state of said subject, and presenting said notification to a user.

29. The method of claim 26, further comprising administering a treatment to said subject for said sleep condition or said sleep disorder, wherein said treatment comprises one or more members selected from the group consisting of administering melatonin, administering a sedative, and administering a sleep therapy.

30. A system for electronically outputting a sleep state of a subject, comprising:
a plurality of sensors comprising at least two members selected from the group consisting of a radar sensor, a thermal sensor, and an audio sensor; and
a computation unit comprising circuitry configured to:
(i) computer process a plurality of signals sensed from a subject using said at least two members selected from the group consisting of said radar sensor, said thermal sensor, and said audio sensor, to generate a latent representation of at least a subset of said plurality of signals;
(ii) generating a fused data set based at least in part on said latent representation generated in (i);
(iii) using a trained algorithm to process said fused data set generated in (ii) to generate a sleep state of said subject; and
(iv) electronically output said sleep state of said subject determined in (iii).
